(19) United States
(12) Patent Application Publication
(10) Pub. No.: US 2023/0046627 A1
(43) Pub. Date: Feb. 16, 2023

(54) FORMIC ACID PRODUCTION METHOD AND FORMIC ACID PRODUCTION SYSTEM

(71) Applicants: IIDA GROUP HOLDINGS CO., LTD., Tokyo (JP); UNIVERSITY PUBLIC CORPORATION OSAKA, Osaka (JP)

(72) Inventors: Kazuhiko Mori, Tokyo (JP); Ayumu Watanabe, Tokyo (JP); Shigeyuki Minami, Osaka (JP); Kunio Kojima, Osaka (JP); Masaki Terada, Osaka (JP)

(21) Appl. No.: 17/792,953

(22) PCT Filed: Jan. 7, 2021
(86) PCT No.: PCT/JP2021/000329

§ 371 Date: Jul. 14, 2022

(30) Foreign Application Priority Data
Jan. 14, 2020 (JP) 2020-003492

(51) Int. Cl. C07C 51/295; B01J 35/00; B01J 21/06; B01J 23/06; B01J 8/06

(52) U.S. Cl. CPC C07C 51/295; B01J 35/004; B01J 21/063; B01J 23/06; B01J 8/065

(57) ABSTRACT

The purpose of the present invention is to provide a formic acid production method and a formic acid production system with high production efficiency and in low cost. It is a formic acid production method comprising: preparing a mixed solution by mixing a solution containing an organic substance with a metal oxide powder having a photocatalyst function; and producing a formic acid by irradiating a light to the mixed solution. Also, it is a formic acid production system comprising: a raw material charging unit into which a solution containing an organic substance and a metal oxide powder having a photocatalyst function are charged; an artificial photosynthesis reaction unit for reacting a mixed solution of the organic substance and the metal oxide powder by irradiating a sunlight or a light to the mixed solution; and a formic acid recovery unit for recovering a formic acid from the mixed solution after an artificial photosynthesis reaction.

### BACKGROUND OF THE INVENTION

### Field of the Invention

Present invention relates to a formic acid production method and a formic acid production system for forming a formic acid from an organic substance using a sunlight. The present application claims priority based on Japanese Patent Application No. 2020-003492 filed in Japan on Jan. 14, 2020, which are incorporated by reference herein.

### Description of Related Art

An idea of “hydrogen economy” using a hydrogen as a fuel has been proposed before, but it is difficult to say that it has become popular even at present from a point of view of an energy conversion efficiency and a difficulty for conveying and storing a hydrogen. For example, in order to convey a hydrogen with low energy density as a fuel for a motor vehicle, it is necessary to apply high pressure of few hundreds barometric pressure. There is a method to use a liquid hydrogen, but it is necessary to be ultralow temperature, so it is not common. Here, a technology to generate and store a formic acid (HCOOH) as an intermediate substance of a hydrogen source has been researched. A formic acid is a liquid in normal temperature, and has high energy density, so it is excellent as a reserve substance.

For example, in Patent Literature 1, a formic acid generation device wherein in artificial photosynthesis, electron transfer to methyl viologen can be achieved without depriving the excitation energy of the dye molecule, and the hydrogen source can be efficiently converted to formic acid and stored, is proposed.

In other words, the invention relating to Patent Literature 1 is a formic acid generation device for supporting a dye, methyl viologen, and formate dehydrogenase on the porous layer of aluminum oxide fine particles formed on the surface of the substrate. Aluminum oxide fine particles do not deprive the excitation energy of the dye molecule, so the electron transfer to methyl viologen can be achieved efficiently, and the hydrogen source can be converted to formic acid and stored.

However, in the invention relating to Patent Literature 1, it requires coenzyme (methyl viologen), so it was not sufficient in terms of cost and efficiency.

To generate an organic substance with high activity from a relatively stable organic substance by utilizing a sunlight as a natural energy is an important technology that humans should obtain at once in many technical fields from a pharmaceutical manufacture to a transportation fuel or an electric power generation, so an establishment of such artificial photosynthesis method and its efficient yield is aspired.

From such circumstances, it is required to provide means for generating a formic acid, which is a useful chemical substance for humans and can be converted relatively easily to a hydrogen as a chemical energy, using a sunlight energy and in low cost.

Patent Literature 1: JP 2018-117576 A

### SUMMARY OF THE INVENTION

The present invention has been invented considering such circumstances, and the purpose of the present invention is to provide a formic acid production method and a formic acid production system with high production efficiency and in low cost.

One embodiment of the present invention is a formic acid production method comprising: preparing a mixed solution by mixing a solution containing an organic substance with a metal oxide powder having a photocatalyst function; and producing a formic acid by irradiating a light to the mixed solution.

According to one embodiment of the present invention, a formic acid is produced with high production efficiency and in low cost by a minimum required combination of an organic substance and a metal oxide powder having a photocatalyst function.

At this time, in one embodiment of the present invention, a metal oxide may be a titanium oxide or a zinc oxide.

A titanium oxide or a zinc oxide is having an excellent effect as a photocatalyst for producing a formic acid.

Also, in one embodiment of the present invention, the organic substance may include a dye.

By using a dye, a production speed of a formic acid can be improved.

Also, in one embodiment of the present invention, a concentration of the metal oxide powder in the mixed solution may be 8% to 18%, and a concentration of the dye in the mixed solution may be 0.02% to 0.11%.

High production speed of a formic acid can be achieved by setting a concentration of the metal oxide powder and a concentration of the dye to be within the above range respectively.

Also, in one embodiment of the present invention, the mixed solution may contain a carbon powder.

When the carbon powder is used instead of the dye, a similar or more preferable effect can be obtained.

Also, in one embodiment of the present invention, the organic substance may include a plant-derived organic substance.

It will be an eco-friendly formic acid production method by using, for example wasted plants such as fallen leaves or waste woods, as raw materials for producing a formic acid.

Also, in one embodiment of the present invention, a production reaction of a formic acid may be performed by circulating the mixed solution and by irradiating a sunlight to the mixed solution.

In this way, materials necessary for an artificial photosynthesis move in a water while being stirred, so a sunlight will be irradiated uniformly and an efficiency of a formic acid production will be improved.

Other embodiment of the present invention is a formic acid production system comprising: a raw material charging unit into which a solution containing an organic substance and a metal oxide powder having a photocatalyst function are charged; an artificial photosynthesis reaction unit for reacting a mixed solution of the organic substance and the metal oxide powder by irradiating a sunlight or a light to the mixed solution; and a formic acid recovery unit for recovering a formic acid from the mixed solution after an artificial photosynthesis reaction.

According to other embodiment of the present invention, it can be a simple structure, so a formic acid production system in which a maintenance is facilitated is provided in low cost.

Also, at this time, in other embodiment of the present invention, the artificial photosynthesis reaction unit is a cylindrical member in a tube shape or an optional shape which transmits a light, and arranged on a rooftop or a roof of a building, and the artificial photosynthesis reaction is performed by circulating the mixed solution in the cylindrical member.

In this way, for example by arranging the cylindrical member on a rooftop or a roof of a house, a formic acid is produced by using a sunlight in daytime, and further, a hydrogen is generated as an energy from that formic acid.

As explained in the above, according to the present invention, a formic acid production method and a formic acid production system with high production efficiency and in low cost is provided.

### BRIEF DESCRIPTION OF THE DRAWINGS

FIG. 1 is a view illustrating a ratio of a production speed of a formic acid for various organic substance materials.

FIG. 2 is a view illustrating a ratio of a production speed of a formic acid for various organic substance materials.

FIG. 3 is a view comparing a production quantity of a formic acid for various metal oxides.

FIG. 4 is a schematic view of an example of a formic acid production system relating to one embodiment of the present invention.

FIG. 5 is a view illustrating a result of performing a formic acid production by irradiating a visible light for 40 hours in a formic acid production method relating to one embodiment of the present invention.

FIG. 6 is a view illustrating a result of measuring a production quantity of a formic acid between organic substances with a dye and without a dye.

FIG. 7 is a view illustrating a result of researching that a formic acid is produced continuously by supplying a dye intermittently in an artificial photosynthesis method in a formic acid production system relating to one embodiment of the present invention.

FIG. 8 is a view illustrating a production speed of a formic acid when a concentration of a titanium oxide and a concentration of a dye is changed in a formic acid production method relating to one embodiment of the present invention.

FIG. 9 is a view illustrating an effect for using a dye and for using a carbon powder in a formic acid production method relating to one embodiment of the present invention.

FIG. 10 is a view illustrating a difference in an effect by a type of irradiated light when a dye (green dye) is used in a formic acid production method relating to one embodiment of the present invention.

FIG. 11 is a view illustrating a difference in an effect by a type of irradiated light when a carbon powder (activated carbon) is used in a formic acid production method relating to one embodiment of the present invention.

### DETAILED DESCRIPTION OF THE INVENTION

In below, explaining about preferred embodiments of the present invention in detail by referring to drawings. In addition, the present embodiments explained in below are not intended to unjustly limit a content of the present invention described in the claims, and not all of configurations explained in the present embodiments are necessary as means for solving a problem of the present invention.

There are mainly two methods for generating a hydrogen by an artificial photosynthesis. A first method is a method for generating a hydrogen by producing a formic acid by irradiating a light to various materials such as a titanium oxide and a dye applied in a film shape, and by imparting a platinum catalyst or the like to that formic acid, and a second method is a method for generating a hydrogen directly from a water by charging a catalyst to a titanium oxide and by irradiating a sunlight. Both methods are presently in an intensive research and development mainly for a purpose of hydrogen generation as an artificial photosynthesis research.

For example, as Patent Literature 1, a method for producing a formic acid has been devised before by irradiating a sunlight to a photocatalyst of a carbon dioxide, a dye, and a titanium oxide. However, its production quantity is extremely low at this time, and also, a hardly broken expensive material is necessary for a dye, so a highly practical system is required.

From a conventional knowledge, a formic acid was produced by preparing a titanium oxide or the like, a viologen, and a dye as a photocatalyst as a solution or a film, and by irradiating a light to the solution or the film. However, it has been found that a viologen and a dye are not essential materials for this reaction, and that a formic acid is produced by progressing an artificial photosynthesis reaction by mixing an organic substance containing a carbon to a photocatalyst.

In other words, one embodiment of the present invention is a formic acid production method comprising: preparing a mixed solution by mixing a solution containing an organic substance with a metal oxide power having a photocatalyst function; and producing a formic acid by irradiating a light to the mixed solution.

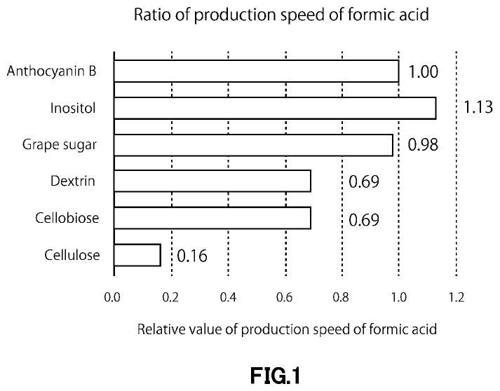

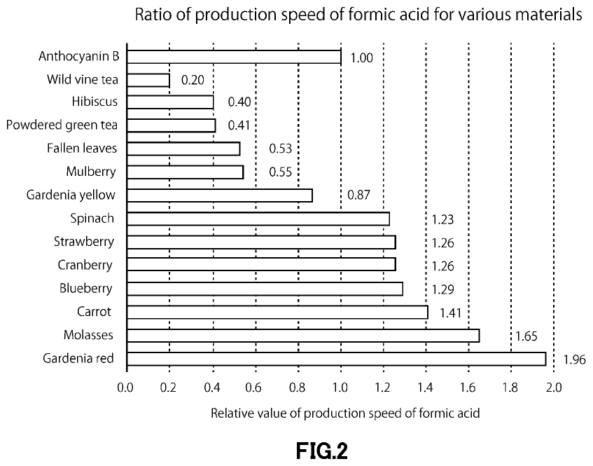

The organic substance is not limited particularly as long as it contains carbon atoms. FIG. 1 and FIG. 2 illustrate a ratio of a production speed of a formic acid for various organic substance materials. The organic substance preferably includes a dye playing a role to assist a photoreaction, but the organic substance without a dye can also be applied. For an example, when setting an anthocyanin B (a dye contained in a violet cabbage) as a reference, as illustrated in FIG. 1, an inositol (a type of vitamin B) and a grape sugar (most existing monosaccharide in nature) are having a ratio of a production speed of a formic acid equivalent to which of an anthocyanin B. Also, a ratio of a production speed of a formic acid is lower than which of an anthocyanin B, but a disaccharide and a polysaccharide such as a dextrin, a cellobiose, and a cellulose can also be applied to the present invention. About saccharides (a monosaccharide, a disaccharide, a polysaccharide), it is preferable to use saccharides having a reducibility.

Also, as illustrated in FIG. 2, the organic substance may be plants, fallen leaves, vegetables, fruits, or the like. By using wasted plants such as fallen leaves or wasted foods such as kitchen garbage as raw materials for producing a formic acid, an eco-friendly formic acid production method can be achieved. When using these waste materials, there is a case that a production quantity and a production speed of a formic acid will be improved by adding an acid or a base accordingly and by performing a heating process. Also, as illustrated in FIG. 2, molasses can be adopted as the organic substance. As molasses, for example waste molasses generated when refining sugar canes or sugar beets can be used effectively.

The organic substance may be a grape sugar only without a dye, but by combining with a dye, a production speed of a formic acid can be improved greatly. As a dye, it has been confirmed that a production speed of a formic acid will be improved greatly by combining with a green dye, a gardenia red, and a mercurochrome, but it may be other dye.

Alternatively, instead of a dye, a carbon powder may be used. As a carbon powder, for example an activated carbon powder is used. As indicated in examples below, by using a carbon powder, a production speed of a formic acid equal to or more than which when a dye is used can be achieved. Also, when using a carbon powder, it has been found that a production speed of a formic acid tends to improve by a light irradiation after irradiating an ultraviolet ray independently.

A production speed of a formic acid also changes according to a concentration of a dye and a metal oxide powder in the mixed solution. It is indicated in examples in below, but as an example, in a formic acid production from a glucose (grape sugar), it is having high production speed of a formic acid when a concentration of a metal oxide powder (titanium oxide) is 8% to 18% and a concentration of a dye (green dye) is 0.02% to 0.11%.

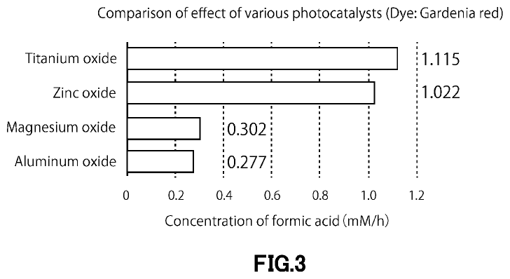

A metal oxide powder is not limited particularly, as long as it is having a photocatalyst function. FIG. 3 is a view comparing a production quantity of a formic acid for various metal oxides. As illustrated in FIG. 3, it is particularly preferable to use a titanium oxide or a zinc oxide. These metal oxides are preferably in a condition of fine particles, in order to increase a contact surface area in the mixed solution. An average particle size of fine particles is not limited particularly, but as an example, it is 20 to 50 nm. Particularly, an average particle size of titanium oxide particles is preferably about 25 nm. Also, among titanium oxides, an anatase type shows an activity about ten times higher than which of a rutile type, so the anatase type is desirable.

A concrete reaction process in the formic acid production method relating to one embodiment of the present invention is not entirely clear, but it is considered as below. At first, as indicated in a reaction formula (1), a water is decomposed by a photocatalyst and an oxygen, hydrogen ions, and electrons are generated.

2H₂O→O₂+4H⁺+4e⁻ (1)

Next, through a photoreaction (artificial photosynthesis) process by a photocatalyst, a dye, or the like, a formic acid is produced from hydrogen ions, electrons, and a carbon dioxide or an organic substance mixed in a solution (a reaction formula (2) or a reaction formula (3) in below). It is characterized in a point that a carbon dioxide is not always required for this reaction. At this time, hydrogen ions and electrons generated in the reaction formula (1) are consumed. Also, a carbon dioxide existing in an atmosphere and/or an exhaust gas from other engines can be used in the reaction formula (2), but it is characterized in that a formic acid can be formed also by a carbon C of the organic substance mixed in the solution in the reaction formula (3).

CO₂+2H⁺+2e⁻HCOOH (2)

Organic substance+2H⁺+2e⁻HCOOH (3)

At this time, among irradiated light, mainly ultraviolet rays act on a metal oxide of a photocatalyst, and visible light act on a dye, so in a formic acid production method relating to one embodiment of the present invention, it is considered that a production efficiency of a formic acid is increased to double by its multifunction. In other words, even in a case of a metal oxide and a dye only, it is considered that electrons can be supplied directly to a formic acid production reaction, by an interaction of a metal oxide and a dye.

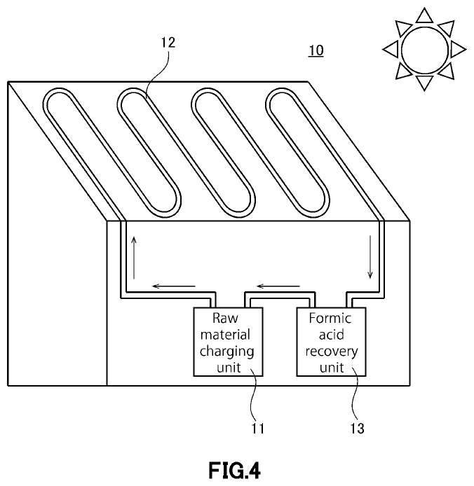

Next, explaining about a formic acid production device relating to one embodiment of the present invention. FIG. 4 a schematic view of an example of a formic acid production system relating to one embodiment of the present invention. One embodiment of the present invention is a formic acid production system 10 comprising: a raw material charging unit 11 into which a solution containing an organic substance and a metal oxide powder having a photocatalyst function are charged; an artificial photosynthesis reaction unit 12 for reacting a mixed solution of the organic substance and the metal oxide powder by irradiating a sunlight or a light to the mixed solution; and a formic acid recovery unit 13 for recovering a formic acid from the mixed solution after an artificial photosynthesis reaction. The raw material charging unit 11 and the formic acid recovery unit 13 may be an identical container.

In the raw material charging unit 11, a solution containing an organic substance and a metal oxide powder having a photocatalyst function are charged. The organic substance and the metal oxide powder may be charged in a mixed condition, or may be charged separately and mixed. The metal oxide powder having a photocatalyst function is scarcely consumed, so after charging the metal oxide powder at first, it is considered to charge only the organic substance additionally as required.

The artificial photosynthesis reaction unit 12 is used as a photochemical reaction device. The artificial photosynthesis reaction unit 12 is preferably configured with a transparent material so that a sunlight or a light by an artificial light source will be irradiated to the mixed solution inside the artificial photosynthesis reaction unit 12, and for example a glass container or a transparent cylindrical member in a tube shape or an optional shape can be cited. Also, in the artificial photosynthesis reaction unit 12, a stirring device or a feeding pump is arranged according to need.

For example, as illustrated in FIG. 4, the artificial photosynthesis reaction unit 12 is a cylindrical member in a tube shape or an optional shape which transmits a light, and arranged on a rooftop or a roof of a building, and an artificial photosynthesis reaction may be performed by circulating the mixed solution in the cylindrical member. By configuring as such, for example by arranging the cylindrical member on a rooftop or a roof of a house, a formic acid is produced by using a sunlight in daytime, and further, a hydrogen is generated as an energy from that formic acid.

In the formic acid recovery unit 13, a formic acid is recovered from the mixed solution after an artificial photosynthesis reaction. A formic acid produced in this way can be stored in a storage facility or the like, for example after concentration.

The formic acid production system relating to one embodiment of the present invention as explained in the above is having advantages as indicated in below.
1. A stirring is not essential, but for high efficiency, a method in which materials necessary for an artificial photosynthesis reaction move in the solution while stirring by using a pump or the like is considered, and in this case, a sunlight will be applied uniformly and it will be highly efficient.
2. A conventional artificial photosynthesis reaction device is contained in a case, so an arrangement in a wide area and a maintenance were not easy, but in one embodiment of the present invention, artificial photosynthesis materials are merely flown for example in a transparent hose as a solution under a sunlight, so even in a wide area, it can be arranged only by extending the hose, so the arrangement is particularly easy.
3. A recovery of produced formic acid and a supplementation of artificial photosynthesis materials can all be performed at a specific place.
4. It can be operated semi-permanently as an artificial photosynthesis device by simply charging and supplying all materials.

### EXAMPLES

In below, it is further explained concretely about the present invention by using examples, but the present invention is not limited in anyway by examples below.

### Example 1

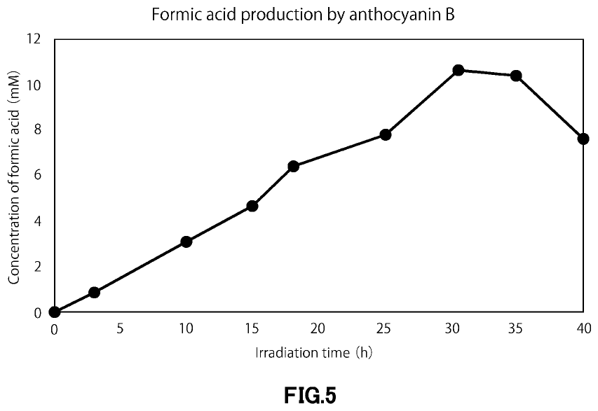

A production quantity of a formic acid was measured according to a lapse of time, by using a titanium oxide powder and an anthocyanin B as a dye, without supplying a carbon dioxide at all, and by irradiating a visible light. A result is illustrated in FIG. 5.

As illustrated in FIG. 5, until 30 hours, a formic acid was produced corresponding to an irradiation time, but from beyond 30 hours, it was confirmed that it will be saturated and the production quantity will be decreased. It is considered that this is because a content of an organic substance contained in an anthocyanin B is limited.

### Example 2

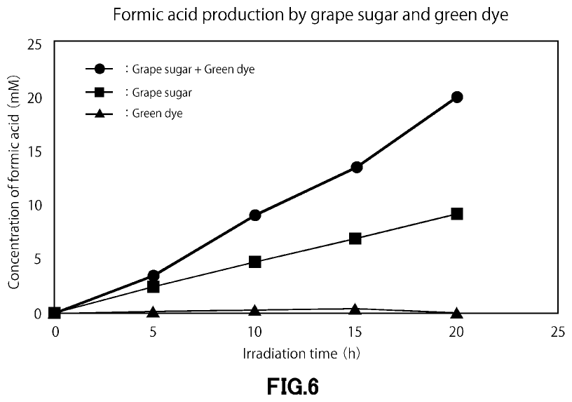

A production quantity of a formic acid was measured by using a dye and an organic substance without a dye, without supplying a carbon dioxide at all. A result of measuring in three patterns of a grape sugar only, a green dye (reagent name: Fast Green) only, and a grape sugar+a green dye is illustrated in FIG. 6.

As illustrated in FIG. 6, it was understood that a formic acid can be produced efficiently by irradiating a light even with a grape sugar without a dye. Also, by mixing a dye to such organic substance other than a dye, a multifunction occurs, and a production quantity of a formic acid was increased to double. In other words, it became clear that a formic acid can be produced in double quantity of a quantity of a formic acid obtained by a grape sugar only, by adding a grape sugar, which is not a dye, to a dye.

### Example 3

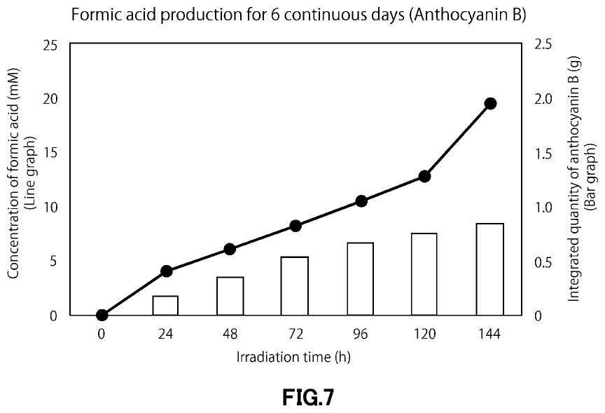

In an experimental condition without supplying a carbon dioxide at all, it was researched about a durability of an artificial photosynthesis method in a formic acid production system. With respect to 2 g of titanium oxide used, a total charged amount of an anthocyanin B was 0.86 g. By adding only an anthocyanin B which is a dye to be a material every day, without adding a titanium oxide, a formic acid production was performed by a light irradiation (HID) for 6 days. A result is illustrated in FIG. 7.

As illustrated in FIG. 7, it became clear that, by applying a formic acid production system relating to the present invention, a formic acid can be produced continuously by charging a dye to be a material intermittently without supplying a titanium oxide, even in an experimental condition without supplying a carbon dioxide at all.

### Example 4

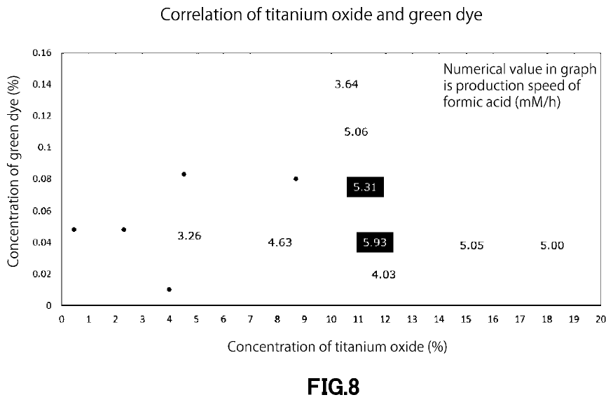

In a mixed solution combining a glucose (grape sugar), a dye (green dye), and a titanium oxide, a production speed of a formic acid was measured by changing a concentration of a dye (green dye) and a titanium oxide respectively. A result is illustrated in FIG. 8.

As illustrated in FIG. 8, about a concentration of a dye (green dye) and a titanium oxide, there is a concentration range most suitable for a production speed of a formic acid, and it was understood that it was having high production speed of a formic acid when a concentration of a metal oxide powder (titanium oxide) in the mixed solution was 8% to 18%, and a concentration of a dye (green dye) in the mixed solution was 0.02% to 0.11%. In addition, highest production speed of a formic acid was achieved when a concentration of a titanium oxide was 12% and a concentration of a green dye was 0.04%.

### Example 5

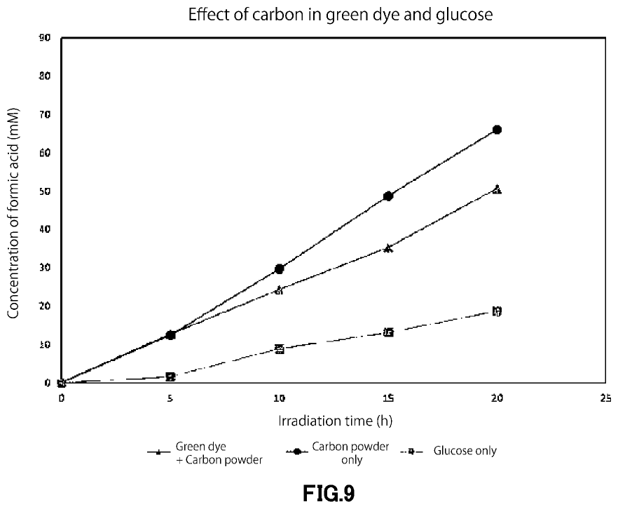

A change with time of a concentration of a produced formic acid was measured for three types of cases using a glucose only, glucose+green dye+carbon powder, and glucose+carbon powder only. A result is illustrated in FIG. 9.

As illustrated in FIG. 9, it was found that, in a case using a carbon powder (activated carbon powder) only, it was having an effect equal to or more than a case when a dye is also added.

### Example 6

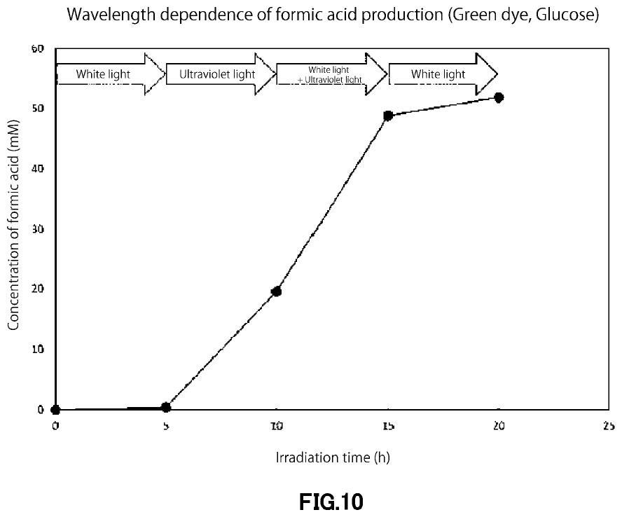

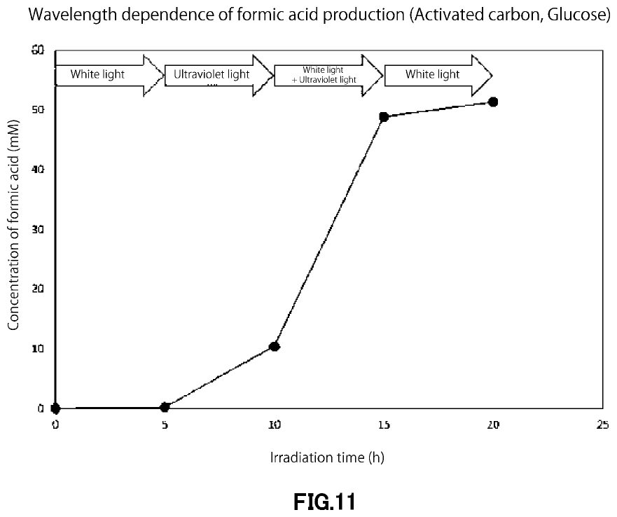

A wavelength dependence of a production speed of a formic acid when a light source (white light, ultraviolet light) is changed was researched for a case using a dye (green dye), and for a case using a carbon powder (activated carbon). In addition, an ultraviolet light was 365 nm which is an absorbing light of a titanium oxide. A result for using a dye (green dye) is illustrated in FIG. 10, and a result for using a carbon powder (activated carbon) is illustrated in FIG. 11.

In both of FIGS. 10 and 11, a production speed of a formic acid was increased when a light source was including an ultraviolet light, and the production speed was increased most with a light of white light+ultraviolet light. Especially, when a carbon powder (activated carbon) was used (FIG. 11), a production speed of a formic acid was improved greatly by a light irradiation after irradiating an ultraviolet light independently.

In addition, it is explained in detail about each embodiment and each example of the present invention as the above, but it can be understood easily for those who skilled in the art that various modifications can be made without practically departing from new matters and effect of the present invention. Therefore, all such variants should be included in the scope of the present invention.

For example, terms described with different terms having broader or equivalent meaning at least once in description and drawings can be replaced with these different terms in any part of description and drawings. In addition, configurations of the formic acid production method and the formic acid production system are not limited to those explained in each embodiment and each example of the present invention, and various modifications can be made.

### Glossary of Drawing References

10 Formic acid production system
11 Raw material charging unit
12 Artificial photosynthesis reaction unit
13 Formic acid recovery unit

1. A formic acid production method comprising:
a mixing step for mixing a solution containing an organic substance with a metal oxide powder having a photocatalyst function;
a reacting step for reacting a mixed solution of the organic substance and the metal oxide powder by irradiating a sunlight or a light to the mixed solution; and
a recovering step for recovering a formic acid from the mixed solution after an artificial photosynthesis reaction,
wherein the mixed solution contains a carbon powder.

2. The formic acid production method according to claim 1, wherein the metal oxide powder is a titanium oxide or a zinc oxide.

3. The formic acid production method according to claim 1, wherein the organic substance includes a dye.

4. The formic acid production method according to claim 1, wherein a concentration of the metal oxide powder in the mixed solution is 8% to 18%, and a concentration of the dye in the mixed solution is 0.02% to 0.11%.

5. (canceled)

6. The formic acid production method according to claim 1, wherein the organic substance includes a plant-derived organic substance.

7. The formic acid production method according to claim 1, wherein a production reaction of a formic acid is performed by circulating the mixed solution and by irradiating a sunlight to the mixed solution.

8. A formic acid production system comprising:
a raw material charging unit into which a solution containing an organic substance and a metal oxide powder having a photocatalyst function are charged;
an artificial photosynthesis reaction unit for reacting a mixed solution of the organic substance and the metal oxide powder by irradiating a sunlight or a light to the mixed solution; and
a formic acid recovery unit for recovering a formic acid from the mixed solution after an artificial photosynthesis reaction,
wherein the mixed solution contains a carbon powder.

9. The formic acid production system according to claim 8, wherein the artificial photosynthesis reaction unit is a cylindrical member in a tube shape or an optional shape which transmits a light, and arranged on a rooftop or a roof of a building, and
the artificial photosynthesis reaction is performed by circulating the mixed solution in the cylindrical member.
